(19) United States
(12) Patent Application Publication
(10) Pub. No.: US 2026/0331230 A1
(43) Pub. Date: Oct. 22, 2026

(54) SUSTAINABLE HAZOP METHODOLOGY, INTEGRATING WITH ARTIFICIAL INTELLIGENCE AND ANALYTIC HIERARCHY PROCESS FOR A SUSTAINABLE CHEMICAL SUBSTITUTION FRAMEWORK

(71) Applicant: The Boeing Company, Arlington, VA (US)

(72) Inventor: Suellen Cristina dos Santos Frank, Lake Stevens, WA (US)

(21) Appl. No.: 19/388,590

(22) Filed: Nov. 13, 2025

(51) Int. Cl. G16C 20/30; G06Q 10/0631; G16C 20/40; G16C 20/70

(52) U.S. Cl. CPC G16C 20/30; G06Q 10/06315; G16C 20/40; G16C 20/70

(57) ABSTRACT

Methods of sustainable integrated chemical substitution assessment are described. A computer implemented method of sustainable integrated chemical substitution assessment includes evaluating a proposed chemical substitution considered sustainable for replacement of an initial chemical using an analytic hierarchy process comprising guideline words selected from at least two sustainability dimensions including social including sustainability, technological including energy and materials, environmental including pollutants, economic including costs, and political including tariffs and embargoes. Systems and computer program products are also described.

### BACKGROUND INFORMATION

### 1. Field

The present disclosure relates generally to chemical substitutions, and more specifically to a novel sustainable hazard and operability study (HAZOP) methodology, integrating with artificial intelligence (AI) and analytic hierarchy process (AHP) for a sustainable chemical substitution framework.

### 2. Background

Hazardous chemicals are deeply embedded in modern industrial society, playing a critical role in the manufacturing of pharmaceuticals, electronics, polymers, pesticides, and countless other products. While these chemicals enable countless advancements, their widespread integration comes with significant, often unaddressed, risks. The inherent properties of many such chemicals, including their persistence in the environment, potential for bioaccumulation, and unforeseen human health and ecotoxicity impacts, pose substantial challenges. A major consequence of these overlooked long-term impacts is the phenomenon of “regrettable substitutions” a problematic chemical is replaced by another that subsequently proves unsuitable due to its own hidden or unforeseen hazards, essentially leading to a “jump out of the frying pan and into the fire” scenario.

A previous approach is Expert Panels: This involves convening groups of experts from relevant fields (e.g., chemistry, toxicology, environmental science, engineering) to discuss and evaluate potential substitutes based on their collective knowledge and experience. Expert judgment has been, and in some contexts still is, a critical part of chemical evaluation, sometimes used to give qualitative or semiquantitative scores when data are scarce. Regulatory bodies also utilize scientific and technical committees comprised of experts to provide advice to regulate a chemical and understand its impact.

A previous approach is Qualitative Scoring Systems: These systems often involve assigning subjective ratings (e.g., high, medium, low) to chemicals or potential substitute is based on various criteria, sometimes informed by expert judgment. These scores might be based on intrinsic properties like toxicity or persistence, or simple estimations of exposure or environmental impact.

A previous approach is Environmental Impact Assessments: These methods broadly evaluate the potential environmental consequences of using a chemical or implementing a substitution. This could range from analyzing specific releases (like in the Toxic Release Inventory (TRI) Environmental Indicators Methodology) to considering broader life cycle aspects, though traditional approaches often focused on discrete stages or impacts rather than a holistic view. Life Cycle Assessment (LCA), is a sophisticated tool that has roots in evaluating environmental impacts throughout a product's life.

A previous approach is Hazard-Based Approaches: A prevalent traditional approach, particularly in earlier regulatory frameworks or initial screening, focuses primarily on the inherent hazardous properties of a chemical (e.g., carcinogenicity, toxicity) regardless of the potential for exposure in a specific use context. While hazard identification is a crucial first step, relying solely on hazard is considered an imprecise tool, as all chemicals have some level of hazard.

A previous approach is Risk-Based Approaches (Traditional): While moving beyond just hazard, traditional risk assessment often focused on calculating risk based on a given level of exposure. However, these traditional risk-based approaches might not adequately account for complexities like long latency periods for health outcomes or the full societal costs of exposure, and the data required for a comprehensive risk assessment for every chemical is often unavailable.

A previous approach is Trial-and-Error Experimentation: Before the advent of systematic design and innovation methodologies, finding substitutes often relied heavily on iterative experimentation in the lab or manufacturing process, testing different chemicals to see which ones performed adequately while hopefully posing fewer apparent issues.

A previous approach is List-Based Screening: A simple method involves checking if a chemical or potential substitute appears on existing regulatory lists of restricted or hazardous substances (e.g., REACH restriction list, SIN-list). This can quickly flag highly hazardous chemicals.

A previous approach is Traditional Toxicology Testing: Evaluating the safety of chemicals has historically relied on traditional in vivo (animal) toxicity tests to identify potential hazards and dose-response relationships. These methods are often time-consuming and expensive.

Consequently, there are ongoing issues with chemical substitution decisions. Methods to evaluate a replacement chemical with an incumbent chemical are complex due to competing criteria, such as, the need to balance performance, toxicity, environmental impact, regulatory compliance, cost and more. Existing tools focus on hazard based identification, risk assessment based on a given level of exposure, trial and error experimentation, list-based screening, and toxicology testing. These methods and tools are often subjective and focus on specific sustainability endpoints. These can require extensive input from the user and often do not provide clear comparison metrics. Traditional methods, including qualitative assessments and environmental impact analyses, fall short in preventing regrettable substitutions. Other issues include accuracy, precision and efficiency with regard to quality, time, and resources.

Therefore, it would be desirable to have a method and apparatus that takes into account at least some of the issues discussed above, as well as other possible issues.

### SUMMARY

Chemical substitution processes face increasing complexity due to competing criteria, such as the need to balance performance, toxicity, environmental impact, regulatory compliance, cost and more. Traditional methods, including qualitative assessments and environmental impact analyses, often fall short in preventing regrettable substitutions and stimulating innovation. To address these challenges, embodiments of this disclosure include an integrated framework combining Artificial Intelligence (AI), the Analytic Hierarchy Process (AHP), and an adapted Hazard and Operability Study (HAZOP) methodology focused on sustainability. The framework leverages AI, as a tool, for predictive toxicology, alternative material discovery, and risk scenario generation; applies AHP for systematic multi-criteria prioritization; and extends HAZOP to incorporate social, technical, environmental, economic, and political risk deviations. A case study in industrial chemical replacement will validate the framework's ability to enhance decision robustness, reduce substitution risks, and improve sustainability performance compared to conventional approaches. Embodiments can deliver a novel tool that adapts to evolving regulations and stakeholder needs, fostering safer and more sustainable chemical innovation.

An embodiment of the present disclosure provides a computer implemented method of sustainable integrated chemical substitution assessment, comprising: evaluating a proposed chemical substitution considered sustainable for replacement of an initial chemical using an analytic hierarchy process comprising guideline words selected from at least two sustainability dimensions including social including sustainability, technological including energy and materials, environmental including pollutants, economic including costs, and political including tariffs and embargoes.

Another embodiment of the present disclosure provides a computer system, comprising: a processor set; a set of one or more computer-readable storage media; program instructions, collectively stored in the set of one or more storage media, for causing the processor set to perform the following computer operations: evaluating a proposed chemical substitution considered sustainable for replacement of an initial chemical using an analytic hierarchy process comprising guideline words selected from at least two sustainability dimensions including social including sustainability, technological including energy and materials, environmental including pollutants, economic including costs, and political including tariffs and embargoes.

Another embodiment of the present disclosure provides a computer program product, comprising: a set of one or more computer-readable storage media; and program instructions, collectively stored in the set of one or more storage media, for causing a processor set to perform the following computer operations: evaluating a proposed chemical substitution considered sustainable for replacement of an initial chemical using an analytic hierarchy process comprising guideline words selected from at least two sustainability dimensions including social including sustainability, technological including energy and materials, environmental including pollutants, economic including costs, and political including tariffs and embargoes.

The features and functions can be achieved independently in various embodiments of the present disclosure or may be combined in yet other embodiments in which further details can be seen with reference to the following description and drawings.

### BRIEF DESCRIPTION OF THE DRAWINGS

The novel features believed characteristic of the illustrative embodiments are set forth in the appended claims. The illustrative embodiments, however, as well as a preferred mode of use, further objectives and features thereof, will best be understood by reference to the following detailed description of an illustrative embodiment of the present disclosure when read in conjunction with the accompanying drawings, wherein:

FIG. 1 is a flowchart illustration of a computer implemented method of sustainable integrated chemical substitution assessment in accordance with an illustrative embodiment;

FIG. 2 is an illustration in the form of a flowchart of a computer implemented method of sustainable integrated chemical substitution assessment in accordance with an illustrative embodiment;

FIG. 3 is an illustration of an aircraft manufacturing and service method in a form of a block diagram in accordance with an illustrative embodiment; and

FIG. 4 is an illustration of an aircraft in a form of a block diagram in which an illustrative embodiment may be implemented.

### DETAILED DESCRIPTION

The illustrative examples recognize and take into account one or more different considerations. The illustrative examples recognize and take into account automated solutions.

This invention provides a system and method for assessing the sustainability of chemicals for hazardous chemical replacement. Embodiments of this disclosure include a system, computer program product, and method for conducting a sustainability assessment for chemicals and/or chemical mixtures, adapting to regulations and stakeholder needs to promote more sustainable chemical substitution decisions. The framework leverages artificial intelligence (AI), as a tool, for predictive toxicology, alternative material discovery, and risk scenario generation; applies analytic hierarchy process (AHP) for systematic multi-criteria prioritization; and extends adapted hazard and operability study (HAZOP) for a holistic approach to incorporate social, technical, environmental, economic, and political risk deviations to generate a qualitative risk matrix as an output.

Embodiments of this disclosure can include an integrated framework combining artificial intelligence (AI). These embodiments can leverage DeepChem to extract hazard and regulatory data for the potential alternatives identified.

Embodiments of this disclosure can include an integrated framework combining analytic hierarchy process (AHP). These embodiments can include a multi-criteria decision-making method that will be used to prioritize chemical alternatives based on multiple criteria.

Embodiments of this disclosure can include an integrated framework combining adapted hazard and operability study (HAZOP) methodology focused on sustainability. Embodiments include developing new sustainability guided questions to assess sustainability related deviations in chemical substitution scenarios.

Thus, embodiments of this disclosure can include an integrated framework combining one, some or all of integrated AI, AHP and/or HAZOP as outlined above.

TABLE 1
Integrated AI + AHP + HAZOP Framework

| | | | Interaction |
|---|---|---|---|
| | | | with Other |
| Methodology | Primary Role | Key Functions | Components |
| Artificial | Data | Semantic search | Provides data inputs |
| Intelligence | synthesis and | and knowledge | and insights for AHP |
| (AI) | predictive | extraction | Supports HAZOP by |
| | analytics | Predictive | identifying potential |
| | | toxicology and | sustainability |
| | | hazard | deviations |
| | | identification | Enhances decision- |
| | | Pattern | making with data |
| | | recognition in | analysis |
| | | sparse data | |
| | | Sustainability | |
| | | deviation | |
| | | detection | |
| Analytic | Structured | Prioritization of | Uses AI-generated |
| Hierarchy | multi- | chemical | data for evaluation |
| Process | criteria | alternatives | Informs HAZOP by |
| (AHP) | decision- | Stakeholder input | ranking alternatives |
| | making | via pairwise | Provides structured |
| | | comparisons | input for |
| | | Sensitivity | expert panels |
| | | analysis | |
| | | and consistency | |
| | | checks | |
| HAZOP | Systematic risk | Lifecycle-based | Uses AHP-ranked |
| (Adapted | identification | deviation | alternatives |
| for | and | analysis | for scenario |
| Sustainability) | sustainability | Use of | analysis |
| | assessment | sustainability | Informed by AI- |
| | | guidewords | identified risks |
| | | Qualitative risk | Provides feedback to |
| | | scoring and | refine AHP and AI |
| | | prioritization | components |

TABLE 2
Qualitative Risk Matrix

| Consequence↓/ | | | |
|---|---|---|---|
| Likelihood→ | Low | Medium | High |
| High | Medium | High | High Toxic |
| | Rare but severe | Unstable supply | substitute |
| | hazard; GC | chain with high | with high |
| | Principle 12; | lifecycle cost; GC | exposure risk; |
| | SDG 3 | Principle 5; | violates GC |
| | | 5DG 9, 12 | Principle |
| | | | 4; SDG 3, 12 |
| Medium | Low | Medium | High |
| | Low toxicity and | Moderate toxicity | Non-renewable |
| | good recyclability; | with partial | feedstock |
| | GC Principle 2; | degradation; GC | with moderate |
| | SDG 12 | Principle 10; | emissions; GC |
| | | SDG 3 | Principle 7; |
| | | | SDG 13 |
| Low | Low | Low | Medium |
| | Fully | Slightly | Minor waste |
| | biodegradable | higher energy | increase |
| | and safe; GC | use; GC Principle | from stable |
| | Principle 10; | 6; SDG 13 | substitute; GC |
| | SDG 12, 13 | | Principle 1; |
| | | | SDG 12 |

In each interior cell of Table 2, the first line indicates low risk, medium risk, or high risk.

Embodiments can include integrating into the AI-AHP-HAZOP framework for transforming chemical risk management. This framework is designed to overcome the limitations of current methodologies by combining one, some or all of the following elements.

Artificial Intelligence (AI), tools that are increasingly being developed to predict the toxicity of new chemicals based on existing data and to screen and design new, safer substitute chemicals. Leveraging tools such as DeepChem to extract, synthesize, and analyze chemical hazard and sustainability data from regulatory and scientific sources. AI will support predictive toxicology, semantic search, and data gap resolution.

Analytic Hierarchy Process (AHP), provides a structured, multi-criteria decision-making framework for evaluating alternatives. It allows for the explicit articulation of decision-makers' preferences and the systematic weighting and comparison of multiple, often conflicting, criteria such as human health and ecotoxicity hazards, broader environmental impacts, and economic and social considerations. This helps manage trade-offs and reduce bias in the assessment process.

HAZOP (Hazard and Operability Study) will systematically identify and analyze risks and sustainability deviations throughout the chemical's lifecycle, from “cradle-to-cradle”. HAZOP is a robust, systematic examination method for identifying hazards and operational problems, applicable to both existing and planned processes, and aids in reducing complexity and focusing on hazard reduction, in this case with a twist implementing sustainability nodes.

Embodiments can improve decision quality, significantly reduce the risk of regrettable substitutions, and support the transition to truly safer, and more sustainable chemical alternatives within industrial processes. By bridging critical gaps in data integration, lifecycle risk assessment, and decision transparency this framework aims to provide a pragmatic, scalable tool for industry and regulators alike for the identification and substitution of hazardous chemicals, thereby enhancing industry competitiveness, public trust, and environmental protection. This study seeks to make a meaningful contribution to knowledge in the field by providing a pragmatic tool to address complex, multi-faceted challenges in chemical management.

Embodiments can include development of a sustainability tailored HAZOP for proactive sustainability risk management. Elements can include one, some or all of the following elements.

An embodiment adapts HAZOP, to develop sustainability-focused dimensions (e.g., social, technological, environmental, economic, and political) and guide words that will support the risk assessment across the lifecycle.

In-depth testing of the new sustainability guided questions to assess sustainability related deviations in chemical substitution scenarios.

Potential of AI support, as a tool, enabling early detection of risks and preventing regrettable substitutions when data is available.

Embodiments can include integration of an AI tool (e.g., DeepChem which is readily commercially available on-line). Embodiments can include one, some or all of the following elements.

Embodiments can use the AI tool to specifically extract hazard and regulatory data for the potential alternatives identified.

The use of this tool will reduce manual workload, enhance decision accuracy, and support dynamic adaptation to evolving regulations and data availability.

Embodiments can include a more structured, objective, and inclusive approach with AI-enhanced AHP.

AHP will be applied with a small, focused panel of 3-5 experts to rank the alternatives from the case studies based on the risk profile.

This integration ensures transparency, reduces bias, and incorporates diverse stakeholder perspectives in evaluating chemical alternatives.

Embodiments can include development of a novel integrated framework for hazardous chemical replacement (HCR).

Embodiments include a novel framework integrating Artificial Intelligence (AI).

The Analytic Hierarchy Process (AHP), and a sustainability-adapted HAZOP methodology to support safer and more sustainable chemical substitution.

By focusing on these three core components, the framework balances innovation with feasibility, offering a structured, data-driven, and risk-aware approach to chemical replacement.

Embodiments can include empirical validation through real-world case studies.

The framework will be applied to one case study in the chemical industry, demonstrating its practical utility and scalability.

Performance will be evaluated against traditional methods using metrics such as sustainability impact, regulatory alignment, and stakeholder satisfaction.

Embodiments can include contribution to advancing sustainable chemistry and global chemical safety.

By addressing the root causes of regrettable substitutions, embodiments of this disclosure support the global transition to safe and sustainable-by-design chemicals.

It aligns with international goals such as SDG (Sustainable Development Goals) such as 12 (Responsible Consumption and Production) and regulatory frameworks like REACH (Registration, Evaluation, Authorisation and Restriction of Chemicals) and TSCA (Toxic Substances Control Act).

Embodiments can include interdisciplinary innovation and future applications.

The framework bridges data science, chemical engineering, sustainability, and risk management, offering a model for cross-disciplinary innovation.

In summary, embodiments can deliver a focused, AI-enhanced decision-support system that empowers industry and regulators to make more informed, transparent, and sustainable chemical substitution decisions, laying the groundwork for a safer and greener future.

HAZOP Adapted for Sustainability is an important aspect of embodiments. Embodiments include evaluating new sustainability guided questions to assess sustainability-related deviations in chemical substitution scenarios. The new methodology will extend the guided words to include sustainability dimensions (e.g., social, technological, environmental, economic, and political). The AI system will assist in enhancing the HAZOP analysis, particularly in identifying potential sustainability deviations, by identifying potential failure modes from within the case studies selected.

Embodiments can leverage semantic search and knowledge extraction. Embodiments can utilize DeepChem to rapidly identify and synthesize information from scientific literature, regulatory databases, and industry reports related to historical sustainability deviations or unforeseen consequences associated with similar chemical classes or applications. This can highlight potential failures modes that might not be immediately apparent to human experts, acting as a proactive brainstorming tool.

Embodiments can include pattern recognition in scarce data. For newly defined sustainability parameters where historical data may be limited or non-standardized, AI models could be employed to identify analogous situations or structural similarities from existing chemical data (even if not explicitly labeled as sustainability deviations) that could indicate potential risks. This ‘fuzzy matching’ capability will generate hypotheses for the expert panel to consider during the HAZOP sessions. The training of AI for this new sustainability parameters will involve leveraging publicly available sustainability databases, qualitative risk assessments from industry reports, and structured expert input provided through the AHP and preliminary HAZOP sessions to build a relevant knowledge base. Table 3 describes the initial definitions and guiding questions.

TABLE 1
Definitions and Guiding Questions HAZOP adapted
for Sustainability

| | Definition in | | | Relevant ACS |
|---|---|---|---|---|
| | the Context | Example HAZOP-Style | | Green |
| Sustainability | of Chemical | Questions (Applying | Relevant UN | Chemistry (GC) |
| Dimension | Substitution | Guidewords) | SDGs | Principals |
| Social | Impacts on | What if there is LESS | SDG 3 (Good | Principle 3: |
| | human well- | public acceptance or MORE | Health and | Less Hazardous |
| | being, | consumer concern | Well-being), | Chemical |
| | including | regarding the substitute | SDG 5 (Gender | Syntheses |
| | worker | chemical family? (What if | Equality), SDG | Principle 12: |
| | health, | local community protests | 10 (Reduced | Inherently |
| | community | arise due to perceived | Inequalities) | Safer |
| | acceptance, | health risks on the new | | Chemistry for |
| | supply chain | substitute?) | | Accident |
| | ethics, and | What if MORE | | Prevention |
| | required | specialized PPE is | | |
| | skillsets. | required? | | |
| | | What if raw materials | | |
| | | are sourced from regions | | |
| | | with OTHER THAN | | |
| | | transparent labor | | |
| | | practices? | | |
| | | What if the substitute | | |
| | | is adopted BEFORE proper | | |
| | | training is implemented? | | |
| | | What if the substitute | | |
| | | ALSO affects downstream | | |
| | | users? | | |
| | | What if the substitute | | |
| | | ALSO introduces new | | |
| | | social risks? | | |
| Technological | Impacts on | What if the substitute | SDG 9 | Principle 6: |
| | manufacturing | requires MORE complex | (Industry, | Design for |
| | processes, | processing equipment? | Innovation, and | Energy |
| | product | What if the substitute | Infrastructure) | Efficiency |
| | performance, | provides PART OF the | | Principle 11: |
| | and potential | required performance but | | Real-time |
| | for future | fails under certain | | analysis for |
| | innovation or | conditions? | | Pollution |
| | obsolescence. | What if adoption of the | | Prevention |
| | | substitute creates a | | |
| | | REVERSE effect, limiting | | |
| | | future upgrades? | | |
| | | What if the substitute | | |
| | | becomes obsolete EARLY in | | |
| | | its lifecycle? | | |
| | | What if it APPLIES TO | | |
| | | legacy systems? | | |
| Environmental | Broader | What if there is LESS | SDG 12 | Principle 1: |
| | lifecycle | biodegradability of the | (Responsible | Prevention |
| | impacts | substitute at the | Consumption and | Principle 5: |
| | beyond | product's end-of-life? | Production), | Safer Solvents |
| | immediate | What if the substitute | SDG 13 (Climate | and |
| | process | requires MORE | Action), SDG 15 | Auxiliaries |
| | safety, | energy/water during its | (Life on Land) | Principle 10: |
| | including | synthesis? | | Design for |
| | resource | What if there is NO | | Degradation |
| | depletion, | viable recycling stream | | |
| | carbon | for the product | | |
| | footprint, | containing the | | |
| | circularity, | substitute? | | |
| | and ecosystem | What if the substitute | | |
| | effects. | ALSO introduces | | |
| | | persistent microplastics? | | |
| | | What if emissions occur | | |
| | | EARLIER in the lifecycle | | |
| | | than expected? | | |
| | | What if the substitute | | |
| | | is used INSTEAD OF a | | |
| | | biodegradable option? | | |
| | | What if the substitute | | |
| | | is derived from NON- | | |
| | | RENEWABLE sources INSTEAD | | |
| | | OF renewable ones? | | |
| Economic | The full | What if the supply | SDG 8 (Decent | Principle 2: |
| | range of | chain for the substitute | Work and | Atom Economy |
| | financial | is LESS stable? | Economic | Principle 9: |
| | impacts | What if the cost of | Growth), SDG 9 | Catalysis |
| | across the | regulatory compliance is | (Industry, | |
| | lifecycle, | MORE than anticipated? | Innovation, and | |
| | including | What if the substitute | Infrastructure) | |
| | price | has OTHER THAN expected | | |
| | volatility, | performance, leading to | | |
| | supply chain | warranty claims? | | |
| | stability, | What if the substitute | | |
| | end-of-life | causes warranty claims | | |
| | liabilities, | AFTER product delivery? | | |
| | and market | What if the substitute | | |
| | competitivene | APPLIES TO a broader | | |
| | Ss. | product line than | | |
| | | intended? | | |
| | | What if the substitute | | |
| | | causes PREMATURE failure, | | |
| | | increasing lifecycle | | |
| | | costs? | | |
| Political / | Risks and | What if a key country | SDG 16 (Peace, | Principle 4: |
| Regulatory | opportunities | enacts MORE stringent | Justice, and | Designing |
| | from | export regulations? | Strong | Safer |
| | government | What if the regulatory | Institutions), | Chemicals |
| | action, | classification of the | SDG 17 | Principle 7: |
| | regulations, | substitute changes in the | (Partnerships | Use of |
| | and | future from ‘non- | for the Goals) | Renewable |
| | geopolitical | hazardous’ to ‘hazardous’ | | Feedstocks |
| | factors. | in a key market, | | |
| | | necessitating costly | | |
| | | changes or re- | | |
| | | authorization? | | |
| | | What if there are NO | | |
| | | harmonized international | | |
| | | regulations for the | | |
| | | substitute? | | |
| | | What if the substitute | | |
| | | is banned BEFORE full | | |
| | | rollout? | | |
| | | What if it APPLIES TO | | |
| | | restricted product | | |
| | | categories? | | |
| | | What if the substitute | | |
| | | is classified as | | |
| | | hazardous INSTEAD OF safe | | |
| | | in another jurisdiction? | | |

To ensure consistency in the qualitative evaluation of the deviations and the assignment of risk scores in Table 2, detailed guidelines for interpreting the severity and likelihood will be developed. This will include concrete examples and illustrative scenarios for each sustainability dimension (social, technological, environmental, economic, and political/regulatory), outlining how specific deviations would translate into ‘Low’, ‘Medium’, or ‘High’ impacts categories. These guidelines will be used during the expert HAZOP sessions to foster a shared understanding and reduce subjectivity in scoring.

Embodiments can include using lifecycle analysis nodes. A hazard and operability study analysis can be structured around key lifecycle stages, which will serve as the ‘nodes’ of the study. The analysis will be systematically applied to each of the following 4 node types. First, Raw Material Sourcing and Transportation: including extraction, procurement, and logistics of raw material. Second, manufacturing and processing: covers all activities related to the production and transformation of materials. Third, product Use and Service Life by the End-User: encompasses the operational phase, including user interaction and performance. Fourth, product End-of-Life (including maintenance, disposal, and recycling): includes maintenance, disposal, recycling, and potential environmental release.

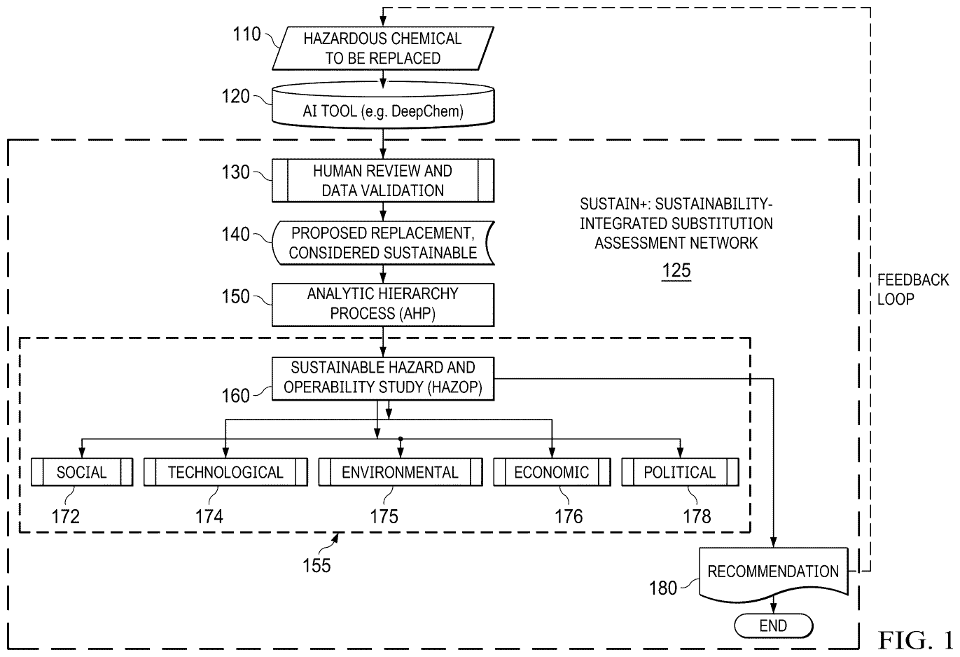

Turning now to FIG. 1, an illustration of a flowchart of a computer implemented method of sustainable integrated chemical substitution assessment is depicted in accordance with an exemplary embodiment. Block 110 includes hazardous chemical to be replaced. Block 120 includes artificial intelligence tool, for example DeepChem software which is readily commercially available on-line. Block 125 includes sustainability-integrated substitution assessment network. Block 130 includes human review and data validation. Block 140 includes proposed replacement, considered sustainable. Block 150 includes analytic hierarchy process (AHP). Block 155 includes evaluating the proposed chemical substitution considered sustainable for replacement of the initial chemical using an analytic hierarchy process comprising guideline words. Block 160 includes sustainable hazard and operability study (HAZOP). Block 172 includes social. Block 174 includes technological. Block 175 includes environmental. Block 176 includes economic. Block 178 includes political. Block 180 includes recommendation. Block 180 is connected via a feedback loop to block 110. Of course, embodiments of this disclosure are not limited to the sequence shown in FIG. 1, or even to these particular elements.

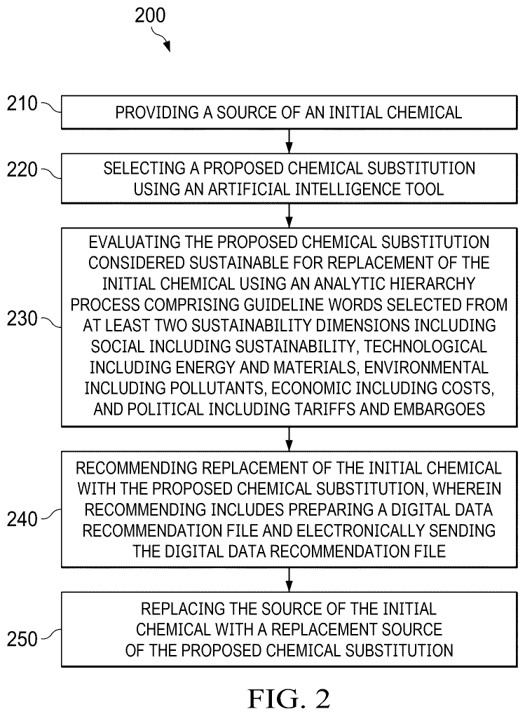

Turning now to FIG. 2, a flowchart of a computer implemented method of sustainable integrated chemical substitution assessment is depicted in accordance with an illustrative embodiment. This method can use the steps shown in FIG. 1. Block 210 includes providing a source of an initial chemical. Block 210 is optional. Block 220 includes selecting a proposed chemical substitution using an artificial intelligence tool. Block 220 is optional. Block 230 includes evaluating the proposed chemical substitution considered sustainable for replacement of the initial chemical using an analytic hierarchy process comprising guideline words selected from at least two sustainability dimensions including social including sustainability, technological including energy and materials, environmental including pollutants, economic including costs, and political including tariffs and embargoes. Block 240 includes recommending replacement of the initial chemical with the proposed chemical substitution, wherein recommending includes preparing a digital data recommendation file and electronically sending the digital data recommendation file. Block 240 is optional. Block 250 includes replacing the source of the initial chemical with a replacement source of the proposed chemical substitution. Block 250 is optional. Of course, embodiments of this disclosure are not limited to the sequence shown in FIG. 2, or even to these particular elements.

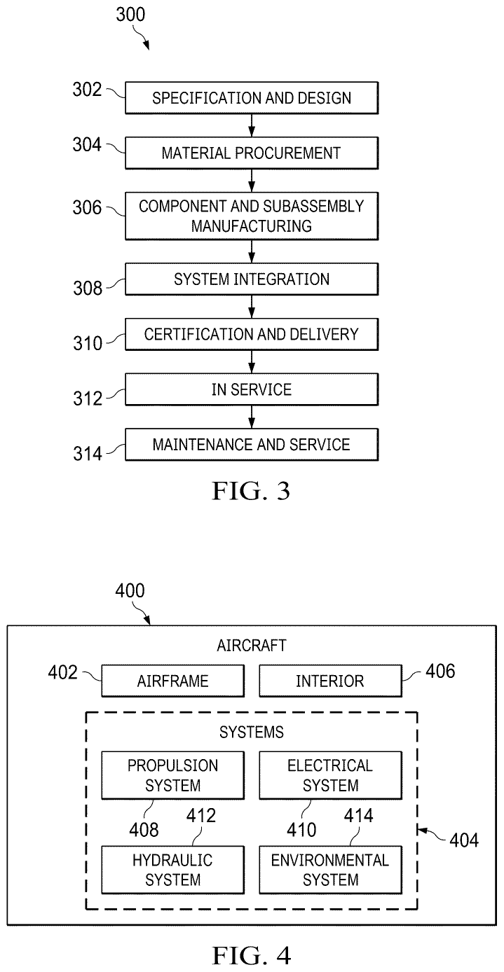

Illustrative embodiments of the present disclosure are described in the context of aircraft manufacturing and service method 300 as shown in FIG. 3 and aircraft 400 as shown in FIG. 4. Turning first to FIG. 3, an illustration of an aircraft manufacturing and service method in a form of a block diagram is depicted in accordance with an illustrative embodiment. During pre-production, aircraft manufacturing and service method 300 may include specification and design 302 of aircraft 400 in FIG. 4 and material procurement 304. Material procurement 304 can include some, most or all of the elements of FIGS. 1-2.

During production, component and subassembly manufacturing 306 and system integration 308 of aircraft 400 takes place. Thereafter, aircraft 400 may go through certification and delivery 310 in order to be placed in service 312. While in service 312 by a customer, aircraft 400 is scheduled for routine maintenance and service 314, which may include modification, reconfiguration, refurbishment, or other maintenance and service.

Each of the processes of aircraft manufacturing and service method 300 may be performed or carried out by a system integrator, a third party, and/or an operator. In these examples, the operator may be a customer. For the purposes of this description, a system integrator may include, without limitation, any number of aircraft manufacturers and major-system subcontractors; a third party may include, without limitation, any number of vendors, subcontractors, and suppliers; and an operator may be an airline, a leasing company, a military entity, a service organization, and so on.

With reference now to FIG. 4, an illustration of an aircraft in a form of a block diagram is depicted in which an illustrative embodiment may be implemented. In this example, aircraft 400 is produced by aircraft manufacturing and service method 300 of FIG. 3 and may include airframe 402 with plurality of systems 404 and interior 406. Examples of systems 404 include one or more of propulsion system 408, electrical system 410, hydraulic system 412, and environmental system 414. Any number of other systems may be included.

Apparatuses and methods embodied herein may be employed during at least one of the stages of aircraft manufacturing and service method 300. One or more illustrative embodiments may be manufactured or used during at least one of component and subassembly manufacturing 306, system integration 308, in service 312, or maintenance and service 314 of FIG. 3.

The illustrations in FIGS. 1-4 are not meant to imply limitations to the manner in which embodiments may be implemented. Other steps in addition to or in place of the ones described and/or illustrated may be used. Also, the steps are presented to illustrate some functional components. One or more of these steps may be combined, divided, or combined and divided into different elements when implemented in an exemplary embodiment.

The flowcharts and block diagrams in the different depicted embodiments illustrate the architecture, functionality, and operation of some possible implementations of methods and apparatuses in an illustrative embodiment. In this regard, each block in the flowcharts or block diagrams may represent at least one of a module, a segment, a function, or a portion of an operation or step.

In some alternative implementations of an illustrative embodiment, the function or functions noted in the blocks may occur out of the order noted in the figures. For example, in some cases, two blocks shown in succession may be executed substantially concurrently, or the blocks may sometimes be performed in the reverse order, depending upon the functionality involved. Also, other blocks may be added in addition to the illustrated blocks in a flowchart or block diagram.

As used herein, the phrase “at least one of,” when used with a list of items, means different combinations of one or more of the listed items may be used and only one of each item in the list may be needed. For example, “at least one of item A, item B, or item C” may include, without limitation, item A, item A and item B, or item B. This example also may include item A, item B, and item C or item B and item C. Of course, any combinations of these items may be present. In other examples, “at least one of” may be, for example, without limitation, two of item A; one of item B; and ten of item C; four of item B and seven of item C; or other suitable combinations. The item may be a particular object, thing, or a category. In other words, at least one of means any combination items and number of items may be used from the list but not all of the items in the list are required. As used herein, “set” when used with reference to items means one or more items. As used herein, “a number of” when used with reference to items means one or more items.

The description of the different illustrative embodiments has been presented for purposes of illustration and description, and is not intended to be exhaustive or limited to the embodiments in the form disclosed. Many modifications and variations will be apparent to those of ordinary skill in the art. Further, different illustrative embodiments may provide different features as compared to other illustrative embodiments. The embodiment or embodiments selected are chosen and described in order to best explain the principles of the embodiments, the practical application, and to enable others of ordinary skill in the art to understand the disclosure for various embodiments with various modifications as suited to the particular use contemplated.

1. A computer implemented method of sustainable integrated chemical substitution assessment, comprising:
evaluating a proposed chemical substitution considered sustainable for replacement of an initial chemical using an analytic hierarchy process comprising guideline words selected from at least two sustainability dimensions including:
social including sustainability,
technological including energy and materials,
environmental including pollutants,
economic including costs, and
political including tariffs and embargoes.

2. The computer implemented method of claim 1, further comprising:
providing, before evaluating, a source of the initial chemical; and replacing, after evaluating, the source of the initial chemical with a replacement source of the proposed chemical substitution.

3. The computer implemented method of claim 1, further comprising:
selecting, before evaluating, the proposed chemical substitution using an artificial intelligence tool.

4. The computer implemented method of claim 1, further comprising:
recommending, after evaluating, replacement of the initial chemical with the proposed chemical substitution, wherein recommending includes preparing a digital data recommendation file and electronically sending the digital data recommendation file.

5. The computer implemented method of claim 1, wherein the analytic hierarchy process comprises guideline words selected from at least three sustainability dimensions including:
social including sustainability,
technological including energy and materials,
environmental including pollutants,
economic including costs, and
political including tariffs and embargoes.

6. The computer implemented method of claim 1, wherein the analytic hierarchy process comprises guideline words selected from at least four sustainability dimensions including:
social including sustainability,
technological including energy and materials,
environmental including pollutants,
economic including costs, and
political including tariffs and embargoes.

7. The computer implemented method of claim 1, wherein the analytic hierarchy process comprises guideline words selected from the sustainability dimensions including:
social including sustainability,
technological including energy and materials,
environmental including pollutants,
economic including costs, and
political including tariffs and embargoes.

8. A computer system, comprising:
a processor set;
a set of one or more computer-readable storage media; and
program instructions, collectively stored in the set of one or more storage media, for causing the processor set to perform the following computer operations:
evaluating a proposed chemical substitution considered sustainable for replacement of an initial chemical using an analytic hierarchy process comprising guideline words selected from at least two sustainability dimensions including:
social including sustainability,
technological including energy and materials,
environmental including pollutants,
economic including costs, and
political including tariffs and embargoes.

9. The computer system of claim 8, wherein the program instructions cause the processor set to perform the following computer operations:
providing, before evaluating, a source of the initial chemical; and replacing, after evaluating, the source of the initial chemical with a replacement source of the proposed chemical substitution.

10. The computer system of claim 8, wherein the program instructions cause the processor set to perform the following computer operations:
selecting, before evaluating, the proposed chemical substitution using an artificial intelligence tool.

11. The computer system of claim 8, wherein the program instructions cause the processor set to perform the following computer operations:
recommending, after evaluating, replacement of the initial chemical with the proposed chemical substitution, wherein recommending includes preparing a digital data recommendation file and electronically sending the digital data recommendation file.

12. The computer system of claim 8, wherein the analytic hierarchy process comprises guideline words selected from at least three sustainability dimensions including:
social including sustainability,
technological including energy and materials,
environmental including pollutants,
economic including costs, and
political including tariffs and embargoes.

13. The computer system of claim 8, wherein the analytic hierarchy process comprises guideline words selected from at least four sustainability dimensions including:
social including sustainability,
technological including energy and materials,
environmental including pollutants,
economic including costs, and
political including tariffs and embargoes.

14. The computer system of claim 8, wherein the analytic hierarchy process comprises guideline words selected from the sustainability dimensions including:
social including sustainability,
technological including energy and materials,
environmental including pollutants,
economic including costs, and
political including tariffs and embargoes.

15. A computer program product, comprising:
a set of one or more computer-readable storage media; and
program instructions, collectively stored in the set of one or more storage media, for causing a processor set to perform the following computer operations:
evaluating a proposed chemical substitution considered sustainable for replacement of an initial chemical using an analytic hierarchy process comprising guideline words selected from at least two sustainability dimensions including:
social including sustainability,
technological including energy and materials,
environmental including pollutants,
economic including costs, and
political including tariffs and embargoes.

16. The computer program product of claim 15, wherein the program instructions cause the processor set to perform the following computer operations:
providing, before evaluating, a source of the initial chemical; and replacing, after evaluating, the source of the initial chemical with a replacement source of the proposed chemical substitution.

17. The computer program product of claim 15, wherein the program instructions cause the processor set to perform the following computer operations:
selecting, before evaluating, the proposed chemical substitution using an artificial intelligence tool.

18. The computer program product of claim 15, wherein the program instructions cause the processor set to perform the following computer operations:
recommending, after evaluating, replacement of the initial chemical with the proposed chemical substitution, wherein recommending includes preparing a digital data recommendation file and electronically sending the digital data recommendation file.

19. The computer program product of claim 15, wherein the analytic hierarchy process comprises guideline words selected from at least three sustainability dimensions including:
social including sustainability,
technological including energy and materials,
environmental including pollutants,
economic including costs, and
political including tariffs and embargoes.

20. The computer program product of claim 15, wherein the analytic hierarchy process comprises guideline words selected from at least four sustainability dimensions including:
social including sustainability,
technological including energy and materials,
environmental including pollutants,
economic including costs, and
political including tariffs and embargoes.
